(19) United States
(12) Patent Application Publication
(10) Pub. No.: US 2011/0308801 A1
(43) Pub. Date: Dec. 22, 2011

(54) Systems, Apparatus and Methods for Extraction of Hydrocarbons From Organic Materials

(76) Inventor: Todd C. Dana, Park City, UT (US)

(21) Appl. No.: 13/049,854

(22) Filed: Mar. 16, 2011

Related U.S. Application Data
(60) Provisional application No. 61/314,471, filed on Mar. 16, 2010.

(51) Int. Cl. E21B 43/24; C07C 7/00; C07C 1/00

(52) U.S. Cl. 166/302; 585/240; 585/800; 166/57

(57) ABSTRACT

A system, apparatus and method for hydrocarbon extraction from organic materials, such as oil shale, coal, lignite, tar sands, animal waste and biomass, which may be characterized generally as feedstock ore. A retort system including at least one fabricated retort vessel may be fabricated within a shaft surrounded by a liner of a process isolation barrier, the upper end of the shaft being closed with a cap sealingly engaged with the liner. The lower end of the shaft provides an exit for collected hydrocarbons, and spent tailings. The shaft may be excavated from the surface into and through one or more subterranean formations, and process control infrastructure is installed within the shaft to for control of hydrocarbon extraction and collection.

### PRIORITY CLAIM

This application claims the benefit of and priority from U.S. Provisional Patent Application No. 61/314,471 filed on Mar. 16, 2010 that is incorporated in its entirety for all purposes by this reference.

### FIELD

Embodiments of the invention relate generally to extraction of hydrocarbons from organic materials and, more specifically, to extraction of hydrocarbons from organic materials in a substantially continuous process employing a vertical retorting system, apparatus employed in the system find associated methods.

### BACKGROUND

Billions of barrels of oil remain locked up in oil shale, coal, lignite, tar sands, animal waste and biomass around the world, yet an economically viable, easily scalable hydrocarbon extraction process has not, to date, been developed. Few, if any, extraction processes are even in commercial use without government subsidies. Throughout the history of unconventional fuel extraction by pyrolysis, many various types of retorting processes have been used, but in general, there are similar genres for these processes. The genres of technologies have generally been categorized as i) above-ground retorts, ii) in-situ processes, iii) modified in-situ processes, and iv) above-ground capsulation processes. Each genre in the prior art exhibits specific benefits, but also associated problems which preclude successful unsubsidized commercial implementation.

### Above-Ground Retorts

Above-ground retorts in the form of fabricated vessels may be of many sizes shapes and designs, offering various attributes in terms of throughput rate, heat recovery, heat source type and horizontal or vertical engineering. Technologies for above-ground retorting include, but are not limited to, plants and facility designs such as those of Petrosix, Fushun, Parahoe, Kiviter and the AlbertaTaciuk Process (ATP), among others. In general, all of these processes are examples of above-ground and fabricated steel retorts which move heated rock through them.

Success of conventional, above-ground retorting has been severely limited due to economic factors. Among the many economic considerations precluding failed commercialization include the cost of fabrication, requiring large volumes of steel, complex forming and welding, compounded by the need to construct ever-larger retorts simply to handle a sufficiently large feedstock ore of hydro carbonaceous material (such as, for example, oil shale) volume to achieve hydrocarbon production on a large-enough scale to justify transportation (pipeline) infrastructure leading to a refinery, or a refinery on site. The perception is that, for retort-based hydrocarbon production on a commercial scale, one must have rapid feedstock ore throughput in order to achieve volume economics; however, any increased feedstock ore throughput must, conventionally also require an increase in heat rate and, therefore, temperature of the overall retort. Yet, by going to a higher retorting temperature, the quality of the produced hydrocarbons decreases and the higher temperature creates a substantially higher volume of emissions than is desirable, or even permissible under ever-mare-restrictive government regulations. Further contributing to the problems of this technology is the requirement for economic viability that the increased heat rate and higher temperature associated with a faster feedstock ore throughput compels the recovery of more energy from the feedstock ore prior to discharge. These energy input and recovery problems associated with conventional retort-based technology are directly related to its poor economic performance.

Another common denominator leading to failure for above-ground retorts is the limitation of retort size. Economically and practically speaking, an above-ground steel retort cannot be built large enough, due to both difficulties in fabrication of a large enough retort vessel as well as required support structure to enable a sufficiently long residence time for feedstock ore at a relatively low temperature to provide adequate throughput. Thus, the limited sizes of above-ground retorts requires a short heating residence time within but, as noted above, the faster, higher heat rate then yields a lower quality oil and greater heat recovery challenge so as not to destroy economics of the process by losing energy efficiency.

### In-Situ Processes

Difficulties relative to limited retort volume from above-ground retort feedstock ore processing gave rise to the concept and development of leaving such hydrocarbonaceous material in place and heating it in formation, such processes being known as “in-sin. processes” and “modified in-situ processes.” The concept of in-situ processes is based on the assumption that by forgoing the mining and handling of feedstock are in favor of drilling through the formation comprising the hydrocarbonaceous material, you can reduce costs by simply introducing heat into the formation through the resulting bore holes to extract hydrocarbon liquids. The logic seems simple and, therefore, sounds like a good idea on paper. Thus, there have emerged many conceptual approaches to introduce heat below ground by drilling a well pattern in the ground and, in some cases, using so-called “intelligent” geometric spacing in an attempt to efficiently add heat or remove gas and liquids.

In-situ processes, while thermally and economically promising in theory, suffer in practice from an undeniable, industry-blocking problem in the form their inability to effectively protect subterranean hydrology proximate the production area following in-situ heating. It is becoming more appreciated with the passage of time and increase in demand due to residential, agricultural, commercial and industrial development that the one natural resource which is more valuable than crude oil is fresh ground water. For example, in oil shale-rich regions around the word- particularly in the Western United States as well as in the deserts of Australia, Jordan and Morocco - fresh water is in limited supply. In some cases, such as in Colorado's Piceance Basin, the oil shale formation is also in direct contact, both above and below, with the fresh water snow pack runoff from the Rocky Mountains.

In recent years several technologies have made progress relating to in-situ recovery, but none have come up with a 100% effective solution for also protecting ground water following in-situ extraction processes. Even with the advent of Royal Dutch Shell's so-called “freeze wall” technology to solidify moisture in-situ surrounding the process area to protect ground water before and during operation of Shell's in-situ process, Shell has not and cannot provide assurance that ground water contamination will not occur after the freeze wall is allowed to thaw. Over time, ground water returns to the formation containing the post-processed materials and then interacts with the formerly heated zones which still contain remaining volatile organic compounds which will then proceed to migrate and eventually contaminate rivers and streams in the area. Confidence related to hydrology protection is therefore needed long after heating of a formation by an in-situ technology. This environmental confidence will only come with the engineered isolation of spent hydrocarbons and ground water, which in-situ processes have been unable to provide.

Another aspect of concern related to in-situ processes is lack of predictability of the overall recovery rate of hydrocarbons from the oil shale or other hydrocarbonaceous material, such as coal, originally in place within the formation. Because in-situ technologies depend on heat introduction methods which hopefully coax hydrocarbons to emerge from production wells, and because subterranean formations are complicated geological structures, there can be no true certainty as to overall recovery rate from an in-situ treated formation. In the case of governments and other entities which lease mineral rights to oil shale or coal producers using such technologies, because royalties paid them are directly related to the overall recovery rate (in terms of volume recovered) of the hydrocarbons in place, recovery in terms of percentage yield of hydrocarbons in place is extremely significant.

### Modified In-Situ Processes

There are many so-called modified in-situ processes employing blasting and even vertical columns in the ground; however, none of these approaches utilize a permeability control infrastructure to collect hydrocarbons or to segregate the rubble zones from the adjacent formation. In other words, a selected portion or a formation containing organic materials is drilled and blasted to create a “rubbleized” area, which may comprise a vertical rubble column. In situ application of heat to, and extraction and collection of hydrocarbons from, the rubbleized material is then effected as described above with respect to traditional in-situ processes.

Both in-situ and modified in-situ hydrocarbon extraction processes may be characterized as “batch” processes, in that organic material containing extractable hydrocarbons is processed in place, i.e., at its site of origin. Therefore, all of the associated infrastructure required for heating the organic material and extracting and collecting hydrocarbons therefrom must be built on site, or transported to the site, and is either left on-site (as in the case of underground components) or, if not worn out during the extraction and collection process, transported to another site for re-use.

### In Capsule Technology

The present inventor is also a named inventor on United States and other patent applications relating to a batch-type hydrocarbon extraction process, which may be characterized herein for convenience as the “in capsule” extraction process. The in capsule extraction process generally relates to the batch extraction of liquid hydrocarbons from hydrocarbonaceous material in the form of a feedstock ore body contained in an earthen impoundment. Relevant to this process are the aspects of heating the impounded hydrocarbonaceous material in place while it is substantially stationary.

Stationary extraction of hydrocarbons is problematic for several reasons. First, the aspect of the feedstock ore remaining substantially stationary, (allowing for only ore movement in the form of vertical subsidence during heating), entails a single use, batch impoundment which is processed until the yield of liquid and volatile hydrocarbons decreases to a point where cost/benefit of energy input to hydrocarbon yield dictates termination of the operation. These impoundments may be envisioned as an array or pattern of very large (in terms of length and width), one use, spread out pads of feedstock ore just below the earth's surface, similar to ore pads employed in a heap leaching process in mining. The width of each such ore pad requires a superimposed vapor barrier to contain hydrocarbon volatiles released during the heating of the feedstock ore to be formed directly on top of, and supported by, the ore body being heated as no structural steel or other separate vapor barrier support span is economically feasible. Thus, the only feasible option of resting the vapor barrier on top of the feedstock ore subjects the vapor barrier to subsidence of the ore as liquid and volatile hydrocarbons are removed.

As subsidence occurs, cracking of the vapor barrier resting on top of the heap also occurs. Further to the problem is that integrity of a clay impoundment barrier such as is designed to prevent release of the hydrocarbon volatiles (i.e., as a vapor barrier), is dependent on retained moisture which is driven off by the process heat. So, as heating occurs over time, not only does subsidence of the feedstock ore increase, but at the same time the clay impoundment dries, until I the lack of underlying support of the clay impoundment in combination with its drying and associated loss of both flexibility and impermeability to hydrocarbon volatiles results in cracking as well as increased porosity. While a polymeric liner may be employed in combination with a clay impoundment vapor barrier in an attempt to stop vapor leakage through cracks in the clay caused by subsidence, the high temperature of gases escaping through the cracks in the clay will come in contact with any such liner and at the high process temperatures employed will likely melt such liner, compromising its integrity. This major problem of vapor barrier compromise as a result of subsidence is highly detrimental to the economics of hydrocarbon recovery, as well as protection of the ambient environment. In other words, a significant percentage, which may exceed 50%, of the potentially recoverable hydrocarbons is lost as escaped volatiles which, in turn, contaminate the atmosphere.

The problem of subsidence of the feedstock ore body also gives rise to other problems associated with operation of the in capsule extraction process. Subsidence may exhibit such a great problem over time that horizontal pipes used to heat the ore body must be protected by significant preplanning to adjust for the sinking of the pipes during heating. In addition, heater pipe penetration joints may be required to anticipate and attempt to mitigate the subsidence issue as a cause of heater pipe collapse and bending under the force of a subsiding ore body above them. It has been proposed to employ corrugated metal pipe as a means to provide heater pipe flexure in tandem with the collapse of the subsiding ore body so as avoid heating pipe breakage. However, none of the foregoing techniques can be used to address heat-induced subsidence, sinking, cracking and integrity compromise or a vapor barrier supported by the impounded feedstock ore body.

The cost to create permeability control infrastructures for each impounded feedstock ore body is another problem from which the in capsule extraction process suffers. Because the in capsule extraction process is applied to an ore body impoundment, there is no “throughput” of the hydrocarbonaceous materials whatsoever, but instead as a batch process requires a new containment barrier for every single batch processed. With substantial preparation and earth work related to clay impoundments or other control liners necessary before hydrocarbons can be extracted from each impounded ore body, the cost of creating an entirely new barrier becomes prohibitive. The in capsule extraction process also entails a heat up period that is costly in terms of energy input and time waiting for heat up to produce a high enough temperature in the ore body for hydrocarbon recovery to commence.

Therefore, because of the problem of barrier cracking as a result of subsidence, the problem of cost associated with continuous barrier and impoundment construction, and because of the heat up requirement of time and energy for each batch, a better, new invention for controlling vapor without risk of barrier cracking and without high cost of barrier construction is needed.

While it should be readily apparent, a disadvantage of any batch-type hydrocarbon extraction process, be it in-situ, modified in-situ or in capsule, is the batch production of the extracted liquid hydrocarbons. When such processes result in production after a period of heating, the large volume of the extracted liquid hydrocarbons produced over a relatively short period of time requires either immediate access to a pipeline for transportation to a refinery or a large storage tank volume, in either case driving up the cost of such an installation.

### SUMMARY

The present invention, in various embodiments, provides straightforward, robust solutions to critical problems associated with conventional hydrocarbon extraction processes applied to hydrocarbonaceous materials (which may also be characterized as organic materials) such as, by way of example and not limitation, feedstock ore (such term being used to encompass organic materials generally, and not limited to mineral or other rock-based materials) in the form of oil shale, coal, lignite, tar sands, animal waste and biomass. Among the advantages offered by implementation of aspects of the present invention are enhanced feedstock ore throughput, superior recovery of hydrocarbon volatiles as well as enhanced environment protection provided by a high-integrity process isolation barrier including an overcap structure supported independently of in-process organic material, lower capital cost achieved through reuse of process and control infrastructure, and better integrity assurance of the final lining of spent (processed) ore tailings due little or no subsidence and associated cracking of a liner placed over a tailings impoundment. Additional advantages include time and cost savings through elimination of repetitive barrier construction associated with batch processing, as well as the requirement of protracted heat up from a cold start for each batch.

Significantly, embodiments of the present invention provide enhanced assurance of volatile hydrocarbon collection from a transportable mass of feedstock ore movable through a laterally geologically supported, such as a subterranean, substantially vertical retort system, integrity of which is not affected by reduction of feedstock ore volume during a heating process employed in hydrocarbon extraction. Embodiments of the invention conduct heating within a descending process and control infrastructure which is supported by at least an adjacent geologic structure, which may be a subterranean formation or formations into which a shaft is excavated. The extraction process employs a process and control infrastructure in the form of a fabricated pass-through retort system disposed within the shaft, and surrounded and capped by a constructed process isolation barrier exhibiting structure integrity independent of reliance upon support by feedstock ore under process. This approach enables maintenance of a substantially continuous process temperature for ongoing hydrocarbon extraction of feedstock ore substantially continuously passing through the retort system without a new heat up period after process temperature has been reached subsequent to system startup, as would be required using a batch processing approach. Only after processed feedstock ore is cooled from the retort system employed in embodiments of the invention and then discharged is such spent ore, which may also be characterized as tailings, transported to a separate, dedicated impoundment area where the relatively cooler and now reduced-volume spent ore will not compromise the integrity of a previously placed and compacted clay liner, or clay or other barrier cap placed thereover for containment and site remediation.

Embodiments of the invention employ substantially continuous volume heating of hydro carbonaceous materials and isolate the heated volume and extraction process from the ambient environment above and surrounding the process site, including ambient atmosphere and adjacent aquifers and, likewise, isolate the process site from encroachment by the ambient environment. Among other things, embodiments of the invention reduce operating costs of hydrocarbon extraction from feedstock ore, while maximizing scalability of processing a moving and heated material, reduce water consumption in processing, assure the avoidance of air and groundwater contamination throughout the entire processing and post-processing handling of feedstock ore, limit surface area disturbance at the processing site, reduce material handling costs, separate fine particulates from the produced oil, and improve hydrogen energy content within the synthetic petroleum liquids, which may be produced from a variety of different feedstock ore sources.

Embodiments of the invention comprise a new and unique genre of pyrolysis, which may be characterized for the sake of convenience, and not by way of limitation, as shaft and (optionally) tunnel pyrolization. System infrastructure is built within the structural confines of a laterally geologically supported liner which both provides structural strength to maintain the shaft opening and enables construction and use of a retort system within the shaft beyond the scale possible with, or even envisioned by, conventional technologies. By utilizing lateral geologic support strength, a massive and scalable retort system, with dimensions and associated volume sufficiently large that, despite constant movement of feedstock ore through the system, can be fabricated, installed and supported within the shaft. Using such a large-volume retort system, the residence time duration of the heated hydro carbonaceous material within the shaft can be maintained for a period of days, requiring relatively much lower temperatures in comparison to higher temperatures employed in conventional retort-based processing with in-retort residence times on the order of minutes, which higher temperatures create more emissions as well as a poorer quality of synthetic fuel.

Embodiments of the invention avoid barrier subsidence and cracking issues associated with the prior art by limiting the horizontal span of a heated containment, while enabling relatively low temperature heating of a large, transported mass of feedstock ore for hydrocarbon extraction, resulting in both high throughput and superior quality of extracted liquid hydrocarbon fuel. In at least one embodiment of the invention, the system is structured for substantially continuous feed of a large volume of feedstock ore through processing to an exit. As a result, high spikes of produced liquid hydrocarbons associated with large, conventional batch processes are avoided, enabling the use of smaller tank farms to handle substantially continuous, more predictable volume liquid hydrocarbon production.

Furthermore, in embodiments of the invention, implementation costs are reduced as the laterally geologically supported liner of the process isolation barrier for the system must be manufactured only once due to the ongoing production of synthetic fuels from the hydrocarbonaceous material passing substantially continuously through the current system.

In one embodiment, the mechanical separation of feedstock ore achieved through crushing may be used to create fine mesh size, high permeability particles which enhance thermal dispersion rates into ore passing through the treatment zone of the system. The added permeability enables the use of low temperatures at long residence times while the particulate or is still moving and falling through the system.

In one embodiment, one or more internal baffle systems may be employed to remove particulates from extracted liquid hydrocarbons.

In one embodiment, easily fabricated and placed vertical heating or cooling conduits in appropriate geometric patterns are situated within the shaft defined by the system liner in conjunction with sensors and open, or preferably closed-loop, valve controlled junctions and heat and cooling sources to yield precise and closely monitored feedstock ore heating and associated vapor and liquid extraction within the treatment zone.

In one embodiment, refractory cement barriers, clay, sand, or gravel liners, steel and gee-membranes typical of engineered shaft structures, or any combination of the foregoing, may be used to construct the laterally geologically supported shaft liner of the process isolation barrier in which the hydrocarbon extraction process takes place.

In one embodiment, temperature and pressure sensors and monitoring mechanisms, fluid dispersion sensors and other richness sensors and data sets combine and input to a computer controlled mechanism with software to optimally control the aspects of the extraction process and manipulate varying gas and liquid extraction compositions in connection with controlling the pass-through flow rate of hydro carbonaceous material.

In one embodiment, insulation can be placed around an entirety, or selected portions of, the perimeter of the shaft for optimized heat containment within the heated treatment zone to reduce required energy input for retorting, and also to protect an adjacent earth formation from adverse effects of the process heat.

In one embodiment, optimal geometric pipe placement for the recovery of heat energy by heat exchange from the moving, heated, processed feedstock ore, may be placed within the lower half of the process isolation barrier and below the heated treatment zone comprising at least one retort vessel and optional associated assemblies, such as a preheat vessel, prior to exit of the ore from the barrier.

In one embodiment, sectioned portions of the process isolation barrier may be constructed in alignment to enable gravity feed of hydro carbonaceous material from upper sections to lower sections and ultimately exited out of the process barrier proximate the bottom thereof. In other words, feedstock ore may be fed by gravity, assisted as necessary or desirable through the use of material transport elements such as, for example, augers, in a controlled manner through the hydrocarbon extraction system to maintain desired temperature and residence time to optimize the quantity and quality of extracted hydrocarbons.

In one embodiment, various temperature zones can be created within the shaft interior of the process isolation barrier for staged and sequenced heating methods, temperatures, gas, fluid and catalyst interactions and thermal transfers. Such interactions can be designed to crack longer chain hydrocarbon chains into lighter fractions within the pyrolyzing process or otherwise combine a portion of fluid or gas reactions within a chamber. This can include the disposition of high pressure chambers within the process isolation barrier to effect some in situ refining of the extracted hydrocarbons. It is also contemplated that the use of a substantially vertical shaft will enable ready partitioning of various temperature zones, so that different hydrocarbon vapors may be drawn off at different temperatures for collection.

In one embodiment, a liner for the lateral perimeter of the process isolation barrier may be created with high temperature cements layered over rebar, steel mesh or wire reinforcements connected to bolts secured in the wall of the excavated perimeter of the shaft excavated for construction of the process isolation barrier. Other liners, such as a fabricated steel liner, may be placed on the interior of such cemented and bolted reinforced liners, as may be free standing clay between two such liners, the clay serving to provide thermal mass to support the hydrocarbon extraction process as well as an effective thermal barrier to contain process heat.

Shaft liners may engineered with, but are not necessarily limited to, liners which include sand, clay, gravel, volcanic ash, spent shale, cement, grout, reinforced cement, refractory cements, insulations, geo-membranes, drainpipes, temperature resistant insulations of penetrating heated pipes, steel liners, corrugated wall liners, shot-crete, rebar, meshes and the like. The shaft liners are used to contain all vapor and liquids created within the treatment zone, and to simultaneously ensure that ground water hydrology does not interact with, or be contaminated by, operations conducted within the process isolation barrier. It is envisioned that the area of the process isolation barrier outside of the outermost liner and within an adjacent formation, may be drained by a drain system adjacent the liner or by additional wells drilled in the formation to limit the amount of underground water in connection with the shaft outer wall or liner.

In one embodiment, gravity assisted hydrocarbon material pass-through mechanisms as known in the art may be utilized to aggregate and channel interior introduction, pathways and exit of such material. Internal gases and fluids, liquids or solvents may also be handled or introduced by any variety of internal pumping, channeling, condensing, heating, staging and discharging, collection, concentrating, piping, and drains, as known in the art.

In one embodiment, hydrocarbon materials of differing composition may be fed into the system for hydrocarbon extraction and exited therefrom through the gravity assisted movement of such materials in any mixed combination or grade or quality of coal, oil shale, tar sands, animal waste or biomass. Optimal compositions and layers or mixes of the foregoing may be introduced into the process isolation barrier, and the system may enable different pass through movement rates, heating rates or residence times for each during the travel through the heated treatment zone. Liquids, chemicals, stabilizers, enzymes, solvents, or catalysts may be used in any variety of ways in the extraction process to optimize or selectively create a desired chemical composition of the gases and fluids being created by heat and or the presence or lack thereof of pressure.

In one embodiment, sections within the gravity assisted shaft treatment zone can be used for placed materials in isolation, in absence of heat, or with intent of limited or controlled combustion or solvent application. Lower content hydrocarbon-bearing material may be useful as a combustion material and used solely for heating other hydrocarbon material passing through the system. In such embodiments, partitioned areas within the process isolation barrier may have oxygen selectively introduced to allow combustion, whereas simultaneously other areas may not have such oxygen or controlled combustion. One example of this may be a shaft pipe within the overall process isolation barrier which actual burns a carbonaceous material to radiate heat. In such instances, such burned material may also be gravity assisted and in a constant state of movement toward the bottom of the process isolation barrier and exit therefrom via a conveyance apparatus through an associated tunnel or other exit means to manage ash, char, charcoal or other by-products of the combustion process. Similarly, such isolated shafts within the process isolation barrier may contain heat transfer fluids, molten salt, or provide for exothermic chemical reactions to create heat or transfer heat to the passing hydrocarbonaceous materials within the system and in proximity to the heating shaft.

In one embodiment, heat from the treatment zone which rises to the top of the shaft enclosed by the process isolation barrier may be redistributed back to the cooler areas of the bottom of the process isolation barrier and or to other, adjacent process isolation barriers housing similar systems. Such heat could be transferred within elevations of the shaft or to other shafts via any number of any type of gas, liquid, heat transfer medium. Such heat may be originally derived from any heat source including, but not limited to, flame less combustors, resistance heaters, natural distributed combustors, nuclear energy, coal energy, fuel cells, solid oxide fuel cells, microwaves or any other type of fuel cell or solar or geothermically derived heat source.

In one embodiment, reducing agents such as hydrogen can be introduced to the treatment zone under pressure and have a desired effect upon the liquids, gases and the hydrocarbonaceous material being processed. More specifically, so-called hydrotreating may be performed in an enclosed chamber within the shaft under pressure (such as 2200-2300 psi) to increase the quality of the extracted hydrocarbons.

In one embodiment, the nature and quality of various fluid and gas compounds included in the extracted products can be altered prior to removal from the extraction system using, as all example, gas-induced pressurization.

Aggregate placements between an internal steel lined shaft and a cemented, reinforced perimeter liner of the process isolation barrier bolted to the formation may be used to act as an insulative barrier. Such aggregates may comprise Bentonite clay or mixtures thereof with spent shale, sand, gravel, aggregates, soil and or volcanic ash. Such an insulative barrier may be equipped with moisture regulation mechanisms to replenish water driven off by the heat from the pyrolyzation process within such barriers on a constant or as-needed basis to maintain adequate moisture in the clay and associated materials.

In one embodiment, the heating rate for the hydrocarbon extraction process is controlled by various methods and adjustments to pressure, heat, and chemical composition of introduced fluids and gases at different elevations. The redistribution of heat can be effected by heat exchangers removing heat toward the bottom of the shaft and redistributing such heat back to a preheater at the top of the shaft proximate the substantially constant feed and gravity induced falling of the hydro carbonaceous material.

In one embodiment, within the process isolation barrier, wells, gathering reservoirs and hardware and various collection and permeable gathering pipes may be placed vertically or horizontally within the process isolation barrier for collection of gases and liquids. Such tubular and non-tubular channels may contain catalysts for creating lighter fractions of hydrocarbon chains being extracted.

In one embodiment, heat within the process isolation barrier may be introduced, controlled and manipulated by mechanical means among various elevations and sections or partitions within the process isolation barrier.

In one embodiment, radio-frequency (RF) mechanisms, solid oxide fuel cells, and other heating devices and emitters may be placed within an interior conduit extending throughout the shaft vertically and mayor may not be mechanically raised and lowered during heating of such devices in effort to distribute or balance heating within the different elevations of the treatment zone.

In one embodiment, sectioned and unitized elevations of the shaft within the greater structured process isolation barrier may be used to transfer, share and balance heat and collect liquids and gases at various elevations to avoid overheating or the need for liquids to migrate through spent shale as it falls via the assistance of gravity within the system toward its exit.

In one embodiment, computer assisted mining, mine planning, hauling, blasting, assay, loading, transport, placement, and dust control measures are utilized to continuously fill and optimize the speed and pass-through rate of mined or harvested hydrocarbonaceous material into and out of the extraction system. Following the exit of the spent hydrocarbonaceous material out of the lower portion of the process isolation barrier through, for example, a tunnel, such material can by conveyed to the surface via a conveyance system which controls off gassing from the material. It is envisioned that a heat quenching and gas squelching or suppressing technique be applied to the spent hydrocarbonaceous upon exit of the spent hydrocarbon material, or “char,” so as to enable its benign introduction to the open atmosphere and placement in a tailings management infrastructure.

In one embodiment, pre-drilling of a pilot bore hole may be used in communication with an intersecting, horizontal tunnel at the bottom of the intended location for the process isolation barrier shaft for the excavation of the shaft via a mechanical, hydraulic excavator to remove formation material.

In one embodiment, substantially precise measurement of weight of the hydrocarbonaceous material may be effected through use of truck or conveyor scales prior to feeding of the material into a process isolation barrier for hydrocarbon extraction. Following extraction of hydrocarbon liquids by pyrolysis within the as the hydrocarbonaceous materials falls to its exit point, the depleted or spent material is again weighed for data and extraction efficiency information. As hydrocarbonaceous material is fed through the shaft and exiting via conveyors through, for example, a connecting tunnel, computers may be used to control the monitoring, heat balancing, gas and fluid extraction measurement, chemical composition and economic modeling of the liquid hydrocarbon product yield in real time.

In one embodiment, blasting, truck and shovel, haul truck transport and dozer leveling is contemplated for mining of hydrocarbonaceous material to be removed from an earth formation at high volume rates to feed the hydrocarbon extraction system within a process isolation barrier.

In one embodiment, combustion of hydrocarbon material may be initiated toward the lower portions of the travel path through the extraction system to create heat for pyrolysis of other hydrocarbonaceous material above such combustion zone within the process isolation barrier.

In one embodiment, fluids can be introduced and circulated through the in-motion gravity falling hydrocarbonaceous material within the shaft to rinse or reduce temperatures to modify various thermal or chemical states of the hydrocarbonaceous materials in process or post-process.

In one embodiment, sodium bi-carbonate and other mineral, precious metal and noble metal leaching solvents, including bioleaching agents, can be introduced within the constructed process isolation barrier to extract metals and minerals from the hydrocarbonaceous materials, particularly but not limited to after hydrocarbon extraction, with or without thermal assistance.

In one embodiment, core drilling, geological reserve analysis and assay modeling of a formation prior to blasting, mining and hauling (or at any time before, after or during such tasks) can serve as data input feeds into computer controlled mechanisms that operate software to identify optimal feed volumes of a system or array of systems within respective process control barriers, and calibrated and cross referenced to desired production rate of liquid hydrocarbons. Example and non-limiting data inputs include, pressurization of the shaft, temperature of the shaft, material input rates, material exit rates, gas weight percentages, gas injection compositions, heating capacity, permeability of the falling hydrocarbonaceous material) material porosity, chemical and mineral composition, moisture content, and hydrocarbons per ton of material. Such analysis and determinations of desirable feed rates and mining rates may include other factors such as weather data factors such as temperature and air moisture content impacting the overall performance of the hydrocarbon extraction system and its inputs and outputs. Other input data such as ore moisture content, hydrocarbon richness, weight, mesh size, and mineral and geological composition may also be utilized as inputs to determine federate and optimum heat residence time, including the time value of money which yields a project cash flow, debt service and internal rates of return for a mine feeding an extraction system comprising one or more process control barriers, each including a hydrocarbon extraction system according to embodiments of the invention.

In one embodiment, mechanisms for treating extracted fluids and gases for the removal of fines and dust particles are envisioned. Separation of fines from shale oil can be a technical challenge and methods to remove impurities can be employed such as, but not limited to, hot gas filtering, centrifuge separation and baffles for liquid particle extraction within the shaft itself.

In one embodiment, final sequestration of CO₂, produced by the heating within the process isolation barrier or combustion therein or for any appurtenant upgrading or refining of the extracted liquid hydrocarbons, or for recycling processes, can be employed. CO₂, sequestration into existing or drilled natural gas or oil wells near the process isolation barrier, once more fully developed as a viable technology, may be employed in tandem with an embodiment of the extraction system of the invention.

In one embodiment, spent oil shale remaining in the shaft treatment zone, if oil shale is employed as feedstock ore, may be utilized in the production of cement and aggregate products for use in the construction or stabilization of the liner walls or to construct additional process isolation barriers for adjacent extraction systems. Such cement products made with the spent shale may include, but are not limited to mixture compositions with Portland cement, calcium, volcanic ash, perlite, synthetic nano-carbons, sand, fiber glass, crushed glass, asphalt, tar, binding resins, cellulosic plant fibers, and more.

In one embodiment, alternative energy sources such as geothermal, solar, wind, wave, biofuels and algae farms derived energy may be incorporated as an external heat source or to create heat for the extraction process.

In one embodiment, various stages of gaseous production may be manipulated through processes which raise or lower temperature and adjust other inputs into the system to produce synthetic gases which can include but are not limited to, carbon monoxide, hydrogen, hydrogen sulfide, hydrocarbons, ammonia, water, nitrogen or various combinations thereof

In one embodiment, hydrocarbonaceous materials may be classified into various grades (such as, for example, hydrocarbon content) and directed into various feedstock shafts disposed within the process isolation barrier for optimized mixing prior to or concurrently with introduction thereof into the treatment zone. For instance, different layers and depths of mined oil shale formations may be richer in certain depth pay zones as they are mined. Once, blasted, mined, shoveled and feed into a shaft as richer oil bearing ores can be bundled or mixed by relative richness of hydrocarbon content for optimal yields or for optimal averaging of the hydrocarbon extraction process within a treatment zone.

In one embodiment, CO₂, emissions from the extraction process may be recovered and used in Enhanced Oil Recovery oil fields which may be adjacent to a hydrocarbon extraction system according to an embodiment of the invention.

In one embodiment, injection, monitoring and production conduits or extraction egresses may be incorporated into any pattern or placement within the process isolation barrier. Monitoring wells within a shaft and even constructed pathways within or adjacent the retort vessel of the treatment zone may be employed to monitor, collect aggregate or control unwanted fluid and moisture migration outside of the retort vessel.

In one embodiment, 3-D, thermal and feed rate software analysis and integrated data input and process simulation may be employed to predict the project economics and outcomes. Computers using software may employ design, operations, optimal extraction methods, and any related process to the extraction system.

In one embodiment, the associated mining or harvesting of hydro carbonaceous material my dictate the placement and location of a process isolation barrier and an appurtenant tunnel for the exit and proper conveyance and handling of spent hydrocarbonaceous material passed through the extraction system.

In one embodiment, surface support equipment such as condensers, pumps, hydrogen plants, gas handling units, electrical supply, heaters, data control and monitoring and valves, sensors and other reusable items may be truck mounted at the surface, within the shaft, or within an exit tunnel adjacent to the process isolation barrier.

In one embodiment, inner liners of the process isolation barrier can be periodically replaced after a suitable amount (in terms of throughput) or period of use of the extraction system or components thereof.

In one embodiment, steel liners may wear out over time and be replaced within the process isolation barrier. Periodic turnaround times wherein all throughput for the extraction system is stopped for maintenance and repair of inner liners, pipes, and other system hardware are contemplated. The use of tungsten carbide liners, hard facing sprays and other wear protection elements and coatings may be used to protect surfaces in contact with falling hydro carbonaceous materials, including but not limited to materials handing mechanisms and shafts, as well as within the retort vessel itself.

In one embodiment, processing of the liquids extracted by the underground shaft retort may be effected to remove particles, nitrogen, sulfur, arsenic, other metals and add hydrogen under pressure. This process is known as “upgrading,” is optional and may or may not be employed to treat the hydrocarbon liquids extracted from the hydrocarbonaceous material.

In one embodiment, the pour point of extracted hydrocarbon liquid is lowered enabling pipeline transportation of highly paraffinic produced products from the process.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 is a schematic, partial side sectional elevation of an embodiment of a hydrocarbon extraction system, including process isolation barrier, according to an embodiment of the invention;

FIG. 1A is shaded, perspective partial side sectional elevation of an embodiment of the invention which may be characterized as a reverse layout of the embodiment depicted in FIG. 1;

FIG. 2 is a schematic, side sectional elevation of a plurality of hydrocarbon extraction systems according to an embodiment of invention employing a common exit tunnel and associated equipment;

FIG. 3 is a schematic of a shaft for a process isolation envelope for a hydrocarbon extraction system according to an embodiment of the invention being excavated and lined with an excavation apparatus;

FIG. 3A is a shaded, perspective partial side sectional elevation corresponding generally to FIG. 3 and depicting additional detail of the of the excavation apparatus and a cable suspension system therefor;

FIG. 4 is a top, schematic elevation of a pattern of multiple process isolation barriers with enclosed extraction systems, two groups thereof each in a linear array and each group substantially aligned with a common exit tunnel; and

FIG. 5 is a schematic side elevation of components of a hydrocarbon extraction system according to an embodiment of the invention.

The drawings are not necessarily to scale.

### DETAILED DESCRIPTION

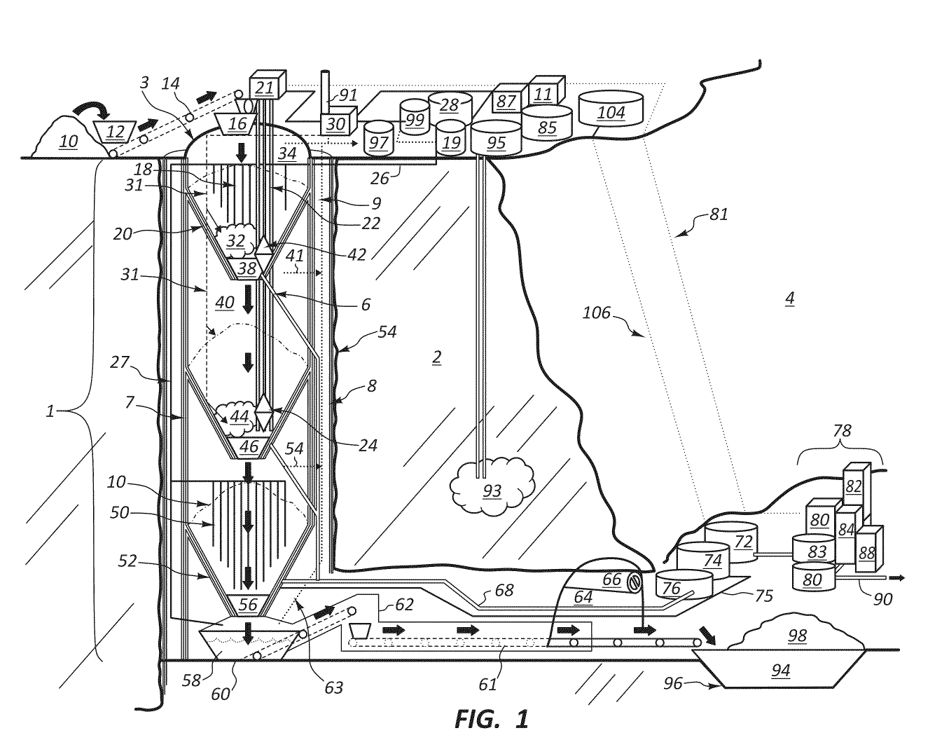

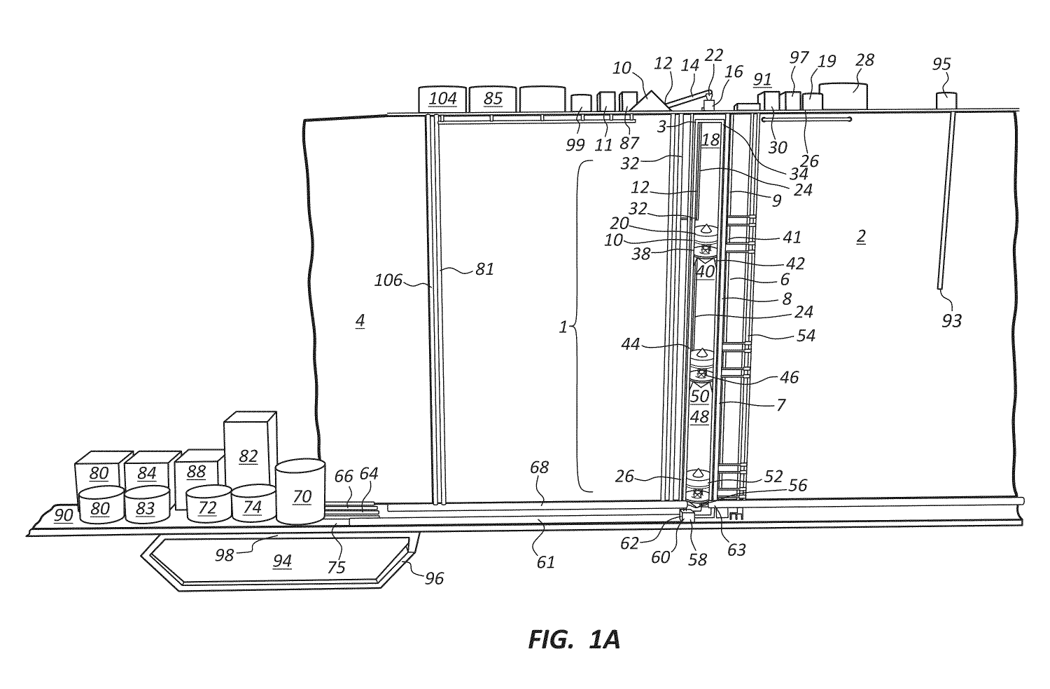

FIG. 1 is a schematic side elevation of an embodiment of the invention facing a cut away formation 2, near a bluff 4 leading to an adjacent area of lower elevation. An excavated shaft exposes an excavated formation face 6. The shaft may have a bore, by way of example, fifty to seventy feet in diameter, and up to several thousand feet in depth. Generally, the shaft may have an aspect ratio, defined as the ratio of shaft length or depth to shaft width or diameter, of at least 1:1. It is contemplated that the aspect ratio, in practice, may be at least 3:1, and aspect ratios of 10:1 and greater are encompassed by the scope of embodiments of the invention.

Support bolts may be inserted into the formation through face 6 to support constructed permeability control infrastructure 7 and 8 comprised of any suitable material or reinforcements for forming a circumferential liner comprising the lateral perimeter of a process isolation barrier for hydrocarbon extraction system 1, which liner acts as to prevent entry into the interior of the process isolation barrier of ground water and gases in the formation 2 as well as to exit of heat and vapors from chambers 20, 40, and 52 of an extraction system 1 of the invention into the formation 2. The hydrocarbon extraction system 1, can, but need not be subdivided to include pre-heat chamber 20, retorting chamber 40, cooling chamber 52, and quench pool 58 as different components within the hydrocarbon extraction system 1. Similarly, more than one pre-heat chamber 20, retorting chamber 40, cooling chamber 52 and quench pool 58 may be disposed within the shaft and operably coupled in either series or parallel, as is desirable based on the nature of the feedstock ore and the intended end product extracted hydrocarbons. Subdividing hydrocarbon extraction system I may be used create separate containments to control vapor, temperature, or through put of organic material in the form of feedstock ore. A top cap 3 of the process isolation barrier comprises a structurally self-supporting, or internally or externally supported, shell spanning the diameter of the shaft contained within the liner of the process isolation barrier as a permeability control infrastructure sealed at its periphery to the permeability control infrastructure comprising liner components 7 and 8 laterally surrounding the hydrocarbon extraction system 1. As such, organic material 10, which may also be characterized as hydrocarbonaceous material or feedstock ore, being introduced into system 1 for hydrocarbon extraction, is free to descend through, and does not support, top cap 3. It is contemplated that this structurally supported, suspended and separate top cap 3, may, but need not be, covered with soil for thermal insulation purposes, conventional insulating materials may be employed, or both.

Stockpiled organic material 10 accumulated from mining, land fill, crop harvest or otherwise is loaded into feed conveyor hopper 12 which feeds organic material onto feed conveyor 14 which, in turn, discharges the organic material 10 into vapor-sealed lock hopper or charge feeder 16. The organic material 10 descends through the vapor sealed lock hopper 16, without any appreciable loss of vapor or heat from within the pre-heating chamber 20. A down hole heat delivery shaft 22 extending downward from control module 21 at the surface into the process isolation barrier, such as through top cap 3, contains a means for delivering heat 24 which may be (by way of non-limiting example) a solid oxide fuel cell, a down hole burner, a microwave generator or other means of delivering heat via the heater shaft 22. The heat rises through the organic material 10 within the pre-heat chamber 20 assisted by the heat from the heat transfer conduits 18 which are fed by an input conduit 26 and its associated manifold and suspended vertically within preheat chamber 20 such that the lithostatic pressure of descending organic material 10 passes by said conduits 26 without damage or significant weight upon them. Other heat transfer conduits 27 are fluidly connected to heat transfer conduits 50 embedded in the descending organic material 10 within cooling chamber 52 to extract heat from the heated organic material 10 by means of heating a heat transfer fluid within the heat transfer conduits 50 circulating upwards to heat transfer conduits 27. A common heat transfer fluid may be circulated in a closed loop within the heat transfer conduits 50, 18, 26 and 27, which may be fluidly connected and may, but need not be, subsidized with additional heat by circulating and heating through an associated heat transfer fluid system 28 or burner/boiler 30. All temperatures of all systems, pipes, facilities, chambers and processes of the subterranean retorting vessel are envisioned to interact with thermal input/output computer control system 11 which manages all federate, discharge, throughput, temperature, data, weights, volumes, liquid amounts, and so forth.

The hydrocarbon extraction system 1 includes other heating means once operational. In addition to heat introduced into chambers 20 and 40 by heat transfer conduits 50 and 26, after start up of operations, as hot vapors are generated during processing of organic material 10 such gases produced from within chambers 20, 40, and 52 exit through gas recovery exits 34, 41, and 54 and are collected by recovery pipes 9. Once collected by recovery pipe 9, these gases may be reheated in burner/boiler 30 to carry subsidized heat back to be re-introduced (recycled) into the bottom of the preheat chamber 20 and/or the bottom of retorting chamber 40 causing a direct gas-to-particle heating of the descending organic material 10.

Alternatively, or in addition to the recovered gases from recovery exits 34, 41 and 54 being utilized as a recycled heating gas, these gases may be introduced to the condenser unit 97 to liquefy a portion of the gases. These condensed liquids from the condenser unit system 97 are fed into the condensed oil tank 104 after passing through an oil-water separator 19. The condensed oil tank 104 may be connected via a pipeline 106 to be combined with produced oil removed from tunnel 64 via gravity-collected oil pipeline 68 and stored in oil tanks 72 for additional storage, or transported elsewhere as desired. The non-condensable hydrocarbons collected in the vapor recovery pipes 9, may alternately be sent for sulfur removal in the gas clean up unit 99. Cleaned gas from the gas cleanup unit 99 may be burned in burner/boiler 30 as a heat source for retorting within chambers 20 or 40 or may be used for other process needs, delivered to chambers 20 and/or 40 by the down hole heat delivery shaft 22, or delivered in a heated state via the recycle gas injection pipe 31 as a hot recycle gas 32 and 44 which rises through organic material 10 within chambers 20 and/or 40. Excess gas from burner/boiler 30 may, optionally, be flared via flare stack 91 or transported to market or utilized in a power generator 87. Direct heat delivered by the down hole heat delivery shaft 22 will augment heat being provided to chambers 20 and 40 by other heat sources lowered down separate conduits within the down hole heat delivery shaft 22. Other heat deliver means lowered down the down hole heat delivery shaft 22 may include, but are not limited to, solid oxide fuel cells, microwave generators, electric resistance heaters, down hole combustion burners and any other heat delivery means located substantially in the vicinity of positions shown as 24.

Organic material 10 introduced into the process isolation barrier substantially continuously descends through the hydrocarbon extraction system 1, augmented and controlled as necessary or desirable by augers or other material handling mechanisms. Gravity pulls the organic material 10 transported by conveyor 14 through the vapor-sealed, preheater charge feeder/lock hopper 16 into preheater chamber 20, which may also be characterized as a vessel. As the organic material 10 descends through preheater chamber 20, it interacts with, and is heated as a result of contact with, preheater zone rising recycle gases 32, and heat transfer conduits 18. Additionally, heat from down hole heater 22 protected by abrasion liner 42 radiates or is directly delivered into the preheater chamber 20 at various locations, including from lowered heating means 24. After heating in preheater chamber 20, organic material 10 descends into vapor-sealed, retort chamber charge feeder 38. The retort chamber charge feeder 38 maintains thermal, vapor and pressure differences between chambers 20 and 40 such that retorting chamber or vessel 40 (with more hydrocarbon vapors) may be at a lesser pressure than the pressure of preheater chamber 20 so as to isolate rising vapors from one chamber to another, yet allow organic material 10 to descend on a substantially continuous basis.

Within retorting chamber 40, organic material 10 under gravity as direct gas-to-particle heating occurs with rising heated recycle gas 44, heat from down hole heater shaft 22 protected by abrasion liner 42 radiating or directly delivered into the retorting chamber 40 at various shaft elevations, including from lowered heating means 24 positioned as shown. After heating in retorting chamber 40, organic material 10 descends into vapor-sealed, cooling chamber charge feeder 46. The cooling chamber charge feeder 46 maintains thermal, vapor and pressure differences between chambers 40 and 52 such that retorting chamber 40 (with more hydrocarbon vapors) may be less pressure than cooling chamber or vessel 52 so as to prevent vapor or thermal communication from one chamber to another, yet allow organic material 10 to descend on a substantially continuous basis.

Within cooling chamber 52, the organic material 10 descends under gravity as heat is removed by heat transfer conduits 50 vertically arranged so as to allow for moving and descending organic material 10 to the bottom of the cooling chamber 52. Other means of cooling (including steam/water quenching) may be effected within cooling chamber 52 and collected by vapor recovery exit 54. After substantial heat removal from chamber 52, organic material 10 descends into vapor-sealed, quench chamber charge feeder 56 which discharges into quenching chamber 60 and its contained quench water 58. Steam generated from the relatively hot, spent organic material 10 contacting the quench water 58 can be transferred as a heat transfer fluid via thermal transfer conduit 27 or vapor recovery exit 63 as desired. The quenching chamber charge feeder 56 keeps thermal, vapor and pressure differences between chambers 60 and 52 separate. It should be understood that vapor-sealed charge feeders 38, 46 and 56 may all be of designs configured to seal vapor, collect gravity-draining oil and liquids as well as slurries, particles and fines. Particle-containing oil and slurry is pumped from these locations via gravity-collected oil pipe 68 and exits tunnel 64 to oil/water separator 70 and then to oil tank 72. Nitrogen generator 74 may be used to generate inert nitrogen gas to be delivered by nitrogen gas pipe 75 for oxygen purging or cooling in one or more of chambers 52, 60, 40 and 20.

The retorted, spent organic materials 10 quenched by quench water 58 are conveyed on conveyor 61 through tunnel 64 with tunnel ventilation system 66 providing fresh air to the tunnel 64. Also keeping air fresh for workers in the tunnel is a conveyor hood vent 62 which controls any remaining off gassing from organic materials 10 on conveyor 61. As organic materials 10 exit tunnel 64 on conveyor 61, a series of mobile or fixed conveyors (or trucks not shown in this FIG. 1) can conveyor haul spent tailings (organic material) to tailing impoundment 94 with permeability control infrastructure 96 made of any material or combination of materials, and be covered and reclaimed by top soil 98 and re-vegetated. The combination of impoundment, liner, and top soil mayor may not include a lining of compacted Bentonite clay and may include drainage pipes (not shown) to divert water from said tailings impoundments.

The collected gravity oil in tank 72 can be sent by pipeline 76 to a separate or adjacent refinery and upgrader 78. The refinery/upgrader includes, but is not limited to, process equipment including a hydrogen plant 80, a distillation tower 82, a hydro-treater 84, arsenic removal means 83, and nitrogen removal and handling means 88. Further to a refinery are other cracking and reforming processes (not shown) for the production of gasoline. Following upgrading at such a facility, the liquids have improved energy, near zero sulfur and nitrogen content and are ready for shipping to crude oil markets via pipeline 90. Hydrogen plant 80, can send hydrogen via hydrogen pipeline 81 as a fuel to a solid oxide fuel cell lowered down hole heater shaft 22 or provide hydrogen for power generation to a fuel cell within power generator 87 to power all process needs. Carbon dioxide from subterranean retorting vessel 1, combustion burner/boiler 30, refinery 78, hydrogen plant 80 and so forth can be collected via carbon dioxide management system 95 and injected into a well bore as geologically sequestered carbon dioxide 93.

To start the heating process for hydrocarbon extraction, propane or other fuel storage 85, supplies fuel to burner/boiler 30 and to power supply generator 87 for all process boilers 30, blowers (not shown), pumps (not shown), conveyors 61 and 14. As retorting occurs, collected hydrocarbons from the retorting process provide make up fuel and also act as a heat transfer fluid.

FIG. 1A may also be referred to for additional detail with respect to the components and operation thereof depicted in FIG. 1, like elements in FIG. 1A to those of FIG. 1 being identified by like reference numerals.

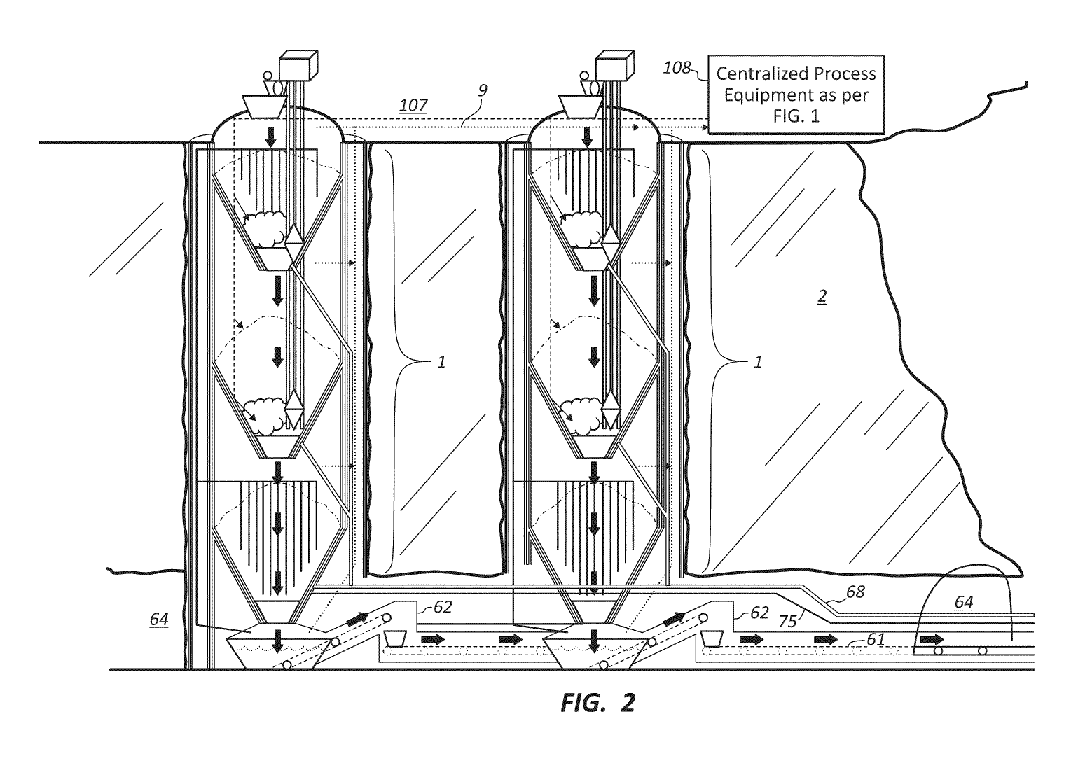

FIG. 2 shows interaction of multiple, subterranean hydrocarbon extraction systems 1 which may comprise as many systems as desired in excavated shafts into formation 2 and aligned with connecting tunnel 64 below. Multiple extraction systems 1 are fluidly connected via recycle gas conduits 107 and vapor recovery pipes 9 as well as power (not shown) and other centralized processing equipment 108 (more fully described in FIG. 1). Organic material 10 is conveyed by conveyor 61 with common conveyor vapor hood 62 from each hydrocarbon extraction system 1. A common tunnel/shaft 64 provides common oil collection 68 and common nitrogen purge lines as well as ventilation (not shown) for both subterranean hydrocarbon extraction systems 1.

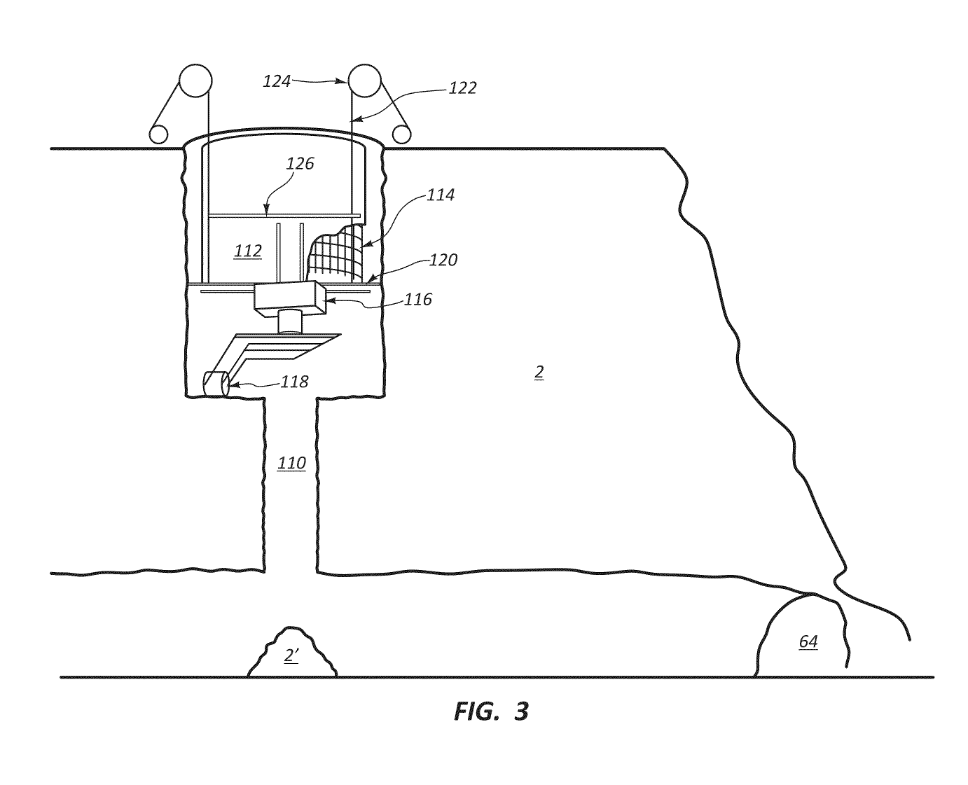

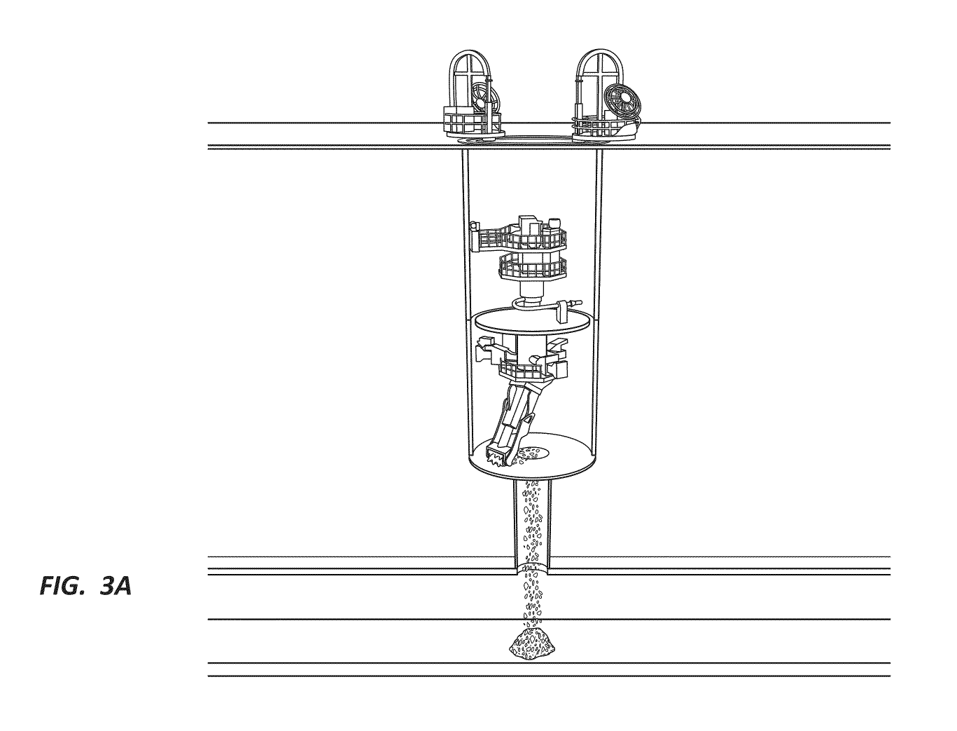

FIG. 3 shows a pre-drilled, core hole in formation 2 expanded to a larger diameter excavation materials exit hole 110 expanded finally to a shaft 112 for constructing a liner 114 of process isolation barrier therein by a shaft sinking machine 116 having an excavation arm 118 and supported by side lowering means 120 supported by cables 122 mounted to pulleys 124, or an by overhead support 126. As can be seen, formation material 2 removed by shaft sinking machine 116 may be dropped through exit hole 110 for removal through exit tunnel 64, which has already been excavated.

FIG. 3A depicts additional detail of a cable suspension system and the shaft sinking machine schematically depicted in FIG. 3;

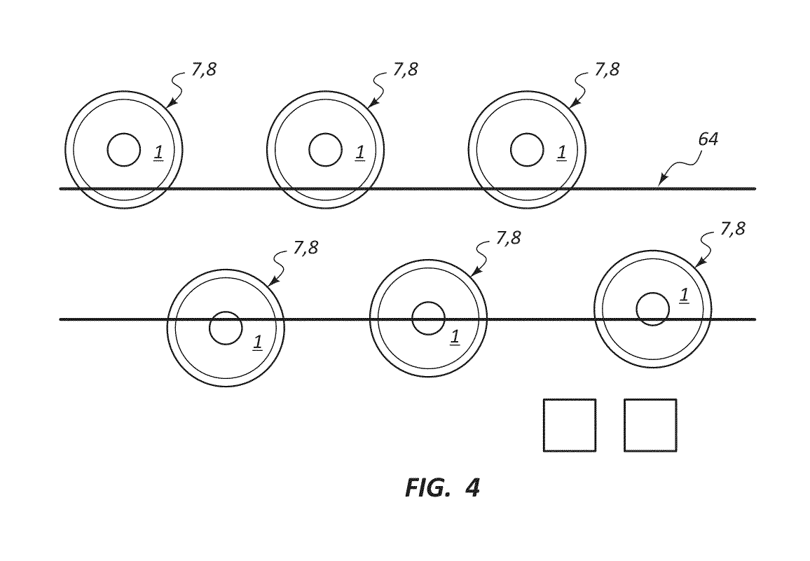

FIG. 4 shows a schematic, top view layout pattern of shafts surrounded by liner components 7, 8 of process isolation barriers of two linearly arranged groups of subterranean hydrocarbon extraction systems 1 employing a common exit tunnel 64 for removal of spent feedstock ore therefrom.

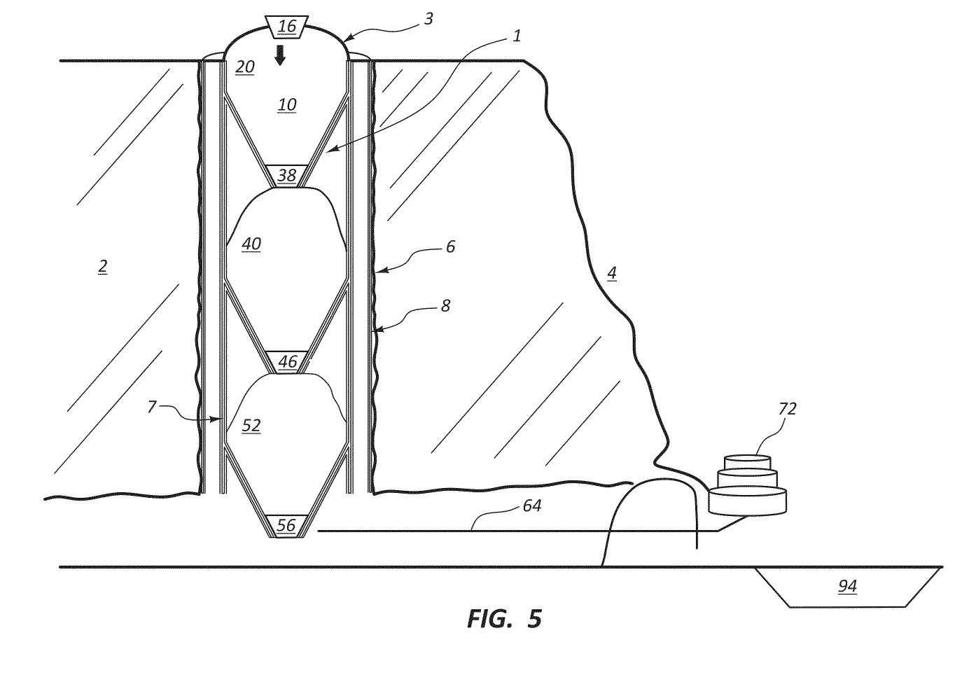

FIG. 5 shows major components of hydrocarbon extraction system 1 as depicted in detail in FIG. 1. Liners 7, 8 surround a shaft covered by top cap 3 and in which are suspended pre-heat chamber 20, retorting chamber 40, and cooling chamber. It can be appreciated that this system arrangement is self-supporting and is not affected by subsidence of the moving hydrocarbonaceous material 10 introduced into system through vapor-sealed lock hopper 16 and movement of this heated feedstock ore as it falls through the shaft from one treatment zone or chamber to another and is transferred between one zone or chamber and another through additional vapor-sealed lock hoppers 38 and 46 before being ejected through vapor-sealed lock hopper 56 into quench pool 58 (not shown), and removed through exit tunnel 64. A plurality of oil tanks 72 comprising a tank farm outside of exit tunnel are used to receive and store extracted liquid hydrocarbons, and spent feedstock ore is deposited in impoundment 94.

Residence time of organic material within a hydrocarbon extraction system of an embodiment of the present invention is contemplated to comprise a time period of between five minutes and ninety-five days, and retorting of the organic material is contemplated to be conducted at a temperature of from about 700° F. to no more than about 1,200° F. and, more specifically, between about 750° F. and 925° F.

It is contemplated that the process isolation barrier may thermally isolate the shaft in which the hydrocarbon extraction process can operate continuously, yet sufficiently reduce high internal temperatures by as much as 400° F. or more, through the barrier to avoid heating outside of the shaft or behind (outside of) the constructed liner of the process isolation barrier, otherwise excessive heating of the adjacent formation may occur, possibly causing vaporization of water in aquifers, other ground water, and any volatiles in the formation surrounding the process barrier. The shaft for may be excavated through at least one geologic formation using a vertical shaft sinking machine. In one embodiment, formation material may be removed from the shaft during excavation thereof downwardly through a smaller pilot hole shaft, which leads to a connecting tunnel, for removal. Alternatively, removed formation material may be pumped to the surface. The shaft may be excavated using a crane-suspended excavator.

The liner for the process isolation barrier to comprise one or more of steel, corrugated pipes, pipes, conduits, rolled steel, clay, Bentonite clay, compacted fill, volcanic materials, refractory cement, cement, synthetic geogrids, fiberglass, rebar, tension cables, nano-carbons, high temperature cement, gabions, meshes, rock bolts, steel anchors, rebar, shot-crete, filled geotextile bags, plastics, cast concrete pieces, wire, cables, polymers, polymer forms, styrene forms, bricks, insulation, ceramic wool, drains, gravel, tar, salt, sealants, pre-cast panels, pre-cast concrete, in-situ concrete, polystyrene forms, steel mats, abrasion resistant materials, tungsten carbide, or combinations thereof

The liner of the process isolation barrier may be fabricated using pre-cast concrete sections lowered vertically down the shaft to form a barrier within the vertical shaft. Such sections may be placed as the shaft is excavated, or subsequent thereto.

The liner of the process isolation barrier may be fabricated to act as a barrier to ground water within an adjacent geological formation, as a barrier to gases within an adjacent geological formation, or both. The liner of the process control barrier may be constructed or placed in direct contact with a wall of the shaft to comprise a barrier between an interior of the process isolation barrier and the face of an adjacent formation.

The top cap of the process isolation barrier spans the shaft and is structurally self-supporting, internally supported or externally supported over an interior of the shaft and is in substantially sealing engagement with the liner of the process isolation barrier. The top cap may be constructed of concrete, steel, cement, reinforcement, mesh, clay, sand, gravel, tension cables, rebar, beams, polyurethane foams, insulations, inflated forms, geodesic steel configurations, or combinations thereof. The top cap may be covered with soil for insulation.

The process isolation barrier may contain reusable structure for passing organic material into and out of the at least one retort vessel for hydrocarbon extraction. The at least one retort vessel may comprises a plurality of conduits disposed within the at least one retort vessel, at least some of the conduits being configured as heating pipes. At least a portion of the plurality of conduits may be oriented substantially vertically.

Feedstock ore may be provided by excavating organic material from a deposit adjacent to the process isolation barrier. Alternatively, the organic material may be sourced from a location remote from the location of the process isolation barrier. The organic material so extracted may be comminuted prior to introduction into the shaft for processing. The organic material may be sized to an approximate particle size of between ¼ inch and 36 inches. The organic material collectively may exhibit a void space of from about 10% to about 50% of a total volume thereof during descent thereof through the process isolation barrier.

To better illustrate the scope of the invention, the organic material may be selected to comprise oil shale, coal, lignite, tar sands, peat, bio mass, wood chips, algae, corn stover, castor plants, sugar cane, hemp plants, used tires, bast fiber family plants, oil sands, tar sands, waste materials, garbage, animal waste, or a combination thereof

The organic material to be processed may be introduced into the at least one retort vessel to descend therein substantially by gravity, for example by use of a vapor sealing lock hopper. The vapor sealing lock hopper may be mounted to the top cap of the process isolation barrier to introduce the organic material therethrough, or may be mounted to the liner of the process isolation barrier or proximate a junction between the top cap and the liner.

Heat energy for hydrocarbon extraction may be provided by combustion of the organic materials, combustion of hydrocarbons, combustion of hydrocarbons removed from the organic material, burners, a solid oxide fuel cell, a fuel cell, waste heat from an adjacent facility, a solar based heat transfer fluid, an electrical resistive heating, solar sources, nuclear power, geothermal, oceanic wave energy, wind energy, a microwave heat source, steam, a super heated fluid, or any combination thereof. If heat energy is created by hydrocarbon combustion, such combustion may be conducted under stoichiometric conditions of fuel to oxygen. If hydrocarbons removed from the organic material are combusted, at least one of sulfur and nitrogen may be removed therefrom prior to combustion. In addition or in the alternative, emissions of carbon monoxide, particle matter, carbon dioxide, nitrous oxide, sulfur dioxins, or combinations thereof may be reduced by employing methods and apparatus known to those of ordinary skill in the art.

Heat for hydrocarbon extraction may be substantially continuously applied, in keeping with the continuous nature of the extraction process, and varied as desired to enhance process conditions.

The application of heat may include injecting heated gases into the at least one retort vessel through which the organic material passes such that the organic material passing through the at least one retort vessel is heated via convection as the organic material descends and heated gases are allowed to pass throughout the retorting vessel. The injected heating gases may be recycled gases recovered from the hydrocarbon extraction, and the recycled gases may be reheated prior to injection into the subterranean retorting vessel.

To enhance processing, the organic material may be heated with elements of a heated, solid material that is separate from the organic material. The elements of heated, solid material may comprise heated sand, heated ceramic balls, hollowed ceramic balls, marbles, organic material containments, heated rocks, heat steel balls, or combinations thereof. The elements of solid material, after heat transfer to the organic material, may be recovered for reheating.

The application of heat may also be effected by transferring heat from a heat transfer fluid through a wall of the process isolation barrier, such as from a conduit within the wall.

The application of heat may also be effected using a plurality of portable combustors, each fluidly connected to a heating conduit embedded within a wall of the process isolation barrier.

The application of heat may comprise heating the organic material sufficiently within a temperature range to substantially avoid formation of carbon dioxide or non-hydrocarbon leachates.

The organic material to be used as feedstock ore may be crushed oil shale, and the application of heat conducted under time and temperature conditions sufficient to form a liquid hydrocarbon product having an API from about 27 to about 45.

The organic material to be used as feedstock ore may be coal, and the application of heat conducted under time and temperature conditions sufficient to form a liquid hydrocarbon product having an API gravity from about 16 to about 35.

The residence time of the organic material within the process isolation barrier may be for a period of between about 5 minutes and 95 days prior to removing the organic material from process isolation barrier.

The application of heat may be thermally controlled by one or more computers, microcontrollers, or other computing means. The thermal control may be used to maintain a substantially continuous temperature of between ambient temperature and about 1200° F.

Extracting hydrocarbons may include purging the extraction environment with an inert gas and, as one non-limiting example, purging the extraction environment may be for the purpose of removing oxygen therefrom.

After hydrocarbon extraction therefrom, removal of organic material from the process isolation barrier may be effected through a vapor sealed lock hopper. Prior to such removal, heat from the organic material may be recovered for reuse in the extraction process, or otherwise.

Heat may be removed from the organic material by introducing heated organic material after the hydrocarbon extraction into a separate cooling chamber vertically positioned below heated elevations (preheat vessel, retort vessel) of the shaft to remove heat from the organic material via means of a heat transfer method. The heat transfer method may comprise the generation of steam, rinsing, air, blowers, heat exchangers, heat transfer fluids, heat transfer conduits, gases, heat transfer conduits with fluidly connected heat transfer fluids, the introduction of solids, heat exchangers, solids to absorb heat, or any combination thereof. Steam generated in the heat transfer method may be used to generate electricity. The transfer of heat, if effected via heat transfer fluids within a conduit connected to the cooling chamber may employ a conduit extending to another chamber within the process isolation barrier.

Further, a heat transfer fluid may be circulated throughout a portion of the shaft beneath a primary heating area such as the preheat vessel or the retort vessel to at least partially recover heat from the organic material.

For some applications, heat within a given shaft may be transferred to another shaft within a second process isolation barrier. Such transfer may be used, for example, to facilitate startup of a hydrocarbon extraction system within the second shaft.

The organic material removal of organic material following the extraction of hydrocarbons therefrom may be accomplished via conveyance through a tunnel proximate and connected to the shaft proximate the lower end thereof. By way of non-limiting example, the tunnel may be excavated using a horizontal boring machine or by room and pillar mining methods. The runnel may be excavated from a location which is a hillside, embankment, cliff, outcrop, ledge or escarpment.

It may be desirable to prevent agglomeration of the organic material at least during the hydrocarbon extraction. By way of non-limiting example, agglomeration may be prevented using chutes, cables, fins, channels, admixes, sizing, mixtures, flutes, beams, riffles, baffles, spirals, ceramic balls, alloy balls, marbles, casings, sonic cavitations, vibratory plates, gases, pressurized gases, vibratory walls, vibration, steel constructions, sand, chimneys, segregation, partitions, screens, meshes, posts, separate chambers, augers, reclaimers or any combination thereof. Means to prevent agglomeration as modular units may be disposed or assembled within the shaft.

If various chambers are used in the process, multiple heating zones may be created. Isolating these chambers may use reclaimer systems which auger organic material above it to lower areas passing such materials through vapor sealed lock hoppers or charge feeders or liquid sealed lock hoppers or charge feeders. It is another embodiment of the invention that liquids falling by gravity to the floors of various chambers would flow away from the direction of solid particles being pulled to the center discharge by a reclaimer or auger.

At least part of the process of hydrocarbon extraction may be performed at above atmospheric pressure. Similarly, at least part of the process of hydrocarbon extraction may be performed below atmospheric pressure.

At least a portion of the retorting vessel interior may be treated with an anti-abrasion protective means. At least a portion of the anti-abrasion means may comprise tungsten carbide.

The process isolation barrier in which the hydrocarbon extraction process is conducted may comprise segregated chambers within the shaft. The segregated chambers may be comprised of preheating chambers, flashing chambers, retorting chambers, combustion chambers, soaking chambers, rinsing chambers, steam chambers, collection chambers, stirring chambers, drying chambers, cooling chambers, heat transfer chambers, loading chambers or any combination thereof.

Conduits for control, heat transfer, extracted hydrocarbon transport, drainage or other purposes may be placed or formed within the liner about the lateral perimeter of the process isolation barrier.

Collection of hydrocarbons removed from the organic material includes cooling the collected hydrocarbons, such as with a condenser. The condenser may be used to separate non-condensable hydrocarbons subsequently used to create heat for the at least one retorting vessel.

Collecting the extracted hydrocarbons may include the extraction of gases at or near the top of the process isolation barrier, the extraction of liquids at two or more elevations within the shaft, or both. The extraction of hydrocarbon liquids at two or more elevations within the process isolation shaft may be employed to mutually segregate at least two of hydrogen, propane, butane, methane, naptha, diesel, distillate, kerosene, residual, or gas oil fractions. The extracted hydrocarbons may be transported from the extraction point using at least one conduit embedded within a wall of the process isolation barrier.

A hydrogen donor agent may be introduced during the hydrocarbon extraction to hydrogenate the hydrocarbons. The hydrogen donor agent may be natural gas, and conditions of pressure and temperature may be maintained sufficient to cause reforming of the hydrocarbons to produce an upgraded hydrocarbon product. As another approach, the extracted hydrocarbons may be collected in a storage vessel to form a body of liquid hydrocarbons and introducing a hydrogen donor agent into the body of liquid hydrocarbons to upgrade the liquid hydrocarbons.

The extracted hydrocarbons may be collected at various elevations within the process isolation barrier, which may include collecting a liquid product from a lower region of the process isolation barrier and collecting a gaseous product from an upper region of the process isolation barrier. At least a portion of the collected gaseous product may be directed to a heat exchanger or other heating apparatus to be heated and recycled through the process isolation barrier one or more times. The recycle gas may be heated to a temperature between 700° F. and 1,200° F.

Carbon dioxide created as a result of application of heat to the organic material may be sequestered by geological sequestration, oceanic sequestration, sequestration into brine liquid, enhanced oil recovery well injection, or combinations thereof. In addition, or as an alternative, a cement additive may be created from the sequestered carbon dioxide in brine liquid.

Organic material collected subsequent to hydrocarbon extraction may be removed from within the process isolation barrier after cooling thereof and placed in an impoundment. The impoundment may comprise an encapsulated infrastructure constructed of steel, corrugated pipes, pipes, conduits, rolled steel, clay, bentonite clay, compacted fill, volcanic materials, refractory cement, cement, synthetic geogrids, fiberglass, rebar, nano-carbon reinforced cement, glass fiber filled cement, high temperature cement, gabions, meshes, rock bolts, rebar, shot-crete, filled geotextile bags, plastics, cast concrete pieces, wire, cables, polymers, polymer forms, styrene forms, bricks, insulation, ceramic wool, drains, gravel, sand, tar, salt, sealants, pre-cast panels, liners, pumps, drains or combinations thereof. The encapsulated infrastructure of the impoundment may be used to provide long term sequestration of the spent organic material from fresh water hydrology, rivers, streams, wildlife, drainages, lakes, plants or combinations thereof

A solvent may be leached through the organic material subsequent to hydrocarbon extraction therefrom, the solvent being a solvent for the extraction of one or more target materials comprising precious metals, noble metals, iron, gold, copper, uranium, aluminum, platinum, nickel, palladium, molybdenum, cobalt, sodium bicarbonate, nacholite, or combinations thereof

The collected, extracted hydrocarbons may be comprised of liquids containing kerogen from oil shale, coal liquids, biomass liquids, oil sands liquids, liquids from lignite, liquids from animal waste, liquids from waste materials, liquids from tires, or combinations thereof

The one or more present inventions, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure.

The present invention, in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for recovering hydrocarbons from organic materials, comprising:
substantially continuously introducing organic material into an upper end of a substantially vertical shaft surrounded by a laterally geologically supported, liner of a process isolation barrier;
extracting hydrocarbons from the organic material by application of heat to the organic material within at least one retort vessel supported within the bore as the organic material moves downward through the shaft;
collecting the extracted hydrocarbons; and
removing organic material from which hydrocarbons have been extracted from proximate a lower end of the shaft.

2. The method of claim 1, further comprising excavating the shaft for through at least one geologic formation using a vertical shaft sinking machine.

3. The method of claim 2, further comprising removing formation material from the shaft downward through a smaller pilot hole shaft, which leads to a connecting tunnel, for removal.

4. The method of claim 2, further comprising pumping excavated formation material to the surface.

5. The method of claim 1, further comprising forming the process isolation barrier to comprise one or more of steel, corrugated pipes, pipes, conduits, rolled steel, clay, bentonite clay, compacted fill, volcanic materials, refractory cement, cement, synthetic geogrids, fiberglass, rebar, tension cables, nano-carbons, high temperature cement, gab ions, meshes, rock bolts, steel anchors, rebar, shot-crete, filled geotextile bags, plastics, cast concrete pieces, wire, cables, polymers, polymer forms, styrene forms, bricks, insulation, ceramic wool, drains, gravel, tar, salt, sealants, pre-cast panels, pre-cast concrete, in-situ concrete, polystyrene forms, steel mats, abrasion resistant materials, tungsten carbide, or combinations thereof.

6. The method of claim 1, further comprising forming the liner of the process control barrier in direct contact with a wall of the shaft to comprise a barrier between an interior of the process isolation barrier and an adjacent formation.

7. The method of claim 1, further comprising excavating organic material from a deposit adjacent to the process isolation barrier.

8. The method of claim 7, further comprising comminuting the organic material prior to introduction into the shaft.

9. The method of claim 1, further comprising selecting the organic material to comprise oil shale, coal, lignite, tar sands, peat, bio mass, wood chips, algae, corn stover, castor plants, sugar cane, hemp plants, used tires, bast fiber family plants, oil sands, tar sands, waste materials, garbage, animal waste, or a combination thereof

10. The method of claim 1, further comprising forming the liner of the process isolation barrier using pre-cast concrete sections lowered vertically down the shaft to form a barrier within the vertical shaft.

11. The method of claim 1, further comprising fabricating the liner of the process isolation barrier to act as a barrier to ground water within an adjacent geological formation.

12. The method of claim 1, further comprising fabricating the liner of the process isolation barrier to act as a barrier to gases within an adjacent geological formation.

13. The method of claim 1, further comprising providing a top cap over the upper end of the process isolation barrier and a floor at the lower end thereof, the top cap being in sealing engagement with a liner defining a lateral perimeter of the process isolation barrier.

14. The method of claim 13, wherein the top cap of the process isolation barrier spans the shaft and is structurally self-supporting, internally supported or externally supported over an interior of shaft and in substantially sealing engagement with the liner of the process isolation barrier.

15. The method of claim 13, further comprising constructing the top cap of concrete, steel, cement, reinforcement, mesh, clay, sand, gravel, tension cables, rebar, beams, polyurethane foams, insulations, inflated forms, geodesic steel configurations, or combinations thereof

16. The method of claim 13, further comprising covering the top cap with soil.

17. The method of claim 1, further comprising moving the organic material introduced into the at least one retort vessel to descend therein substantially by gravity.

18. The method of claim 1, further comprising introducing the organic material into the shaft by use of a vapor sealing lock hopper.

19-93. (canceled)

94. A system for extracting hydrocarbons from organic material, the system comprising:
a substantially vertical shaft defining a bore;
a process isolation barrier liner surrounding the shaft and in contact with the earth along at least a portion of a circumference and along at least a majority of a depth of the shaft;
a top cap extending over an upper end of the bore and comprising support structure suspending the top cap over the bore, a periphery of the top cap in substantially sealed engagement with the process isolation barrier;
apparatus for introducing organic material into the upper end of the bore and configured for preventing substantial escape of vapor from the bore;
at least one fabricated retort vessel supported within the bore; and
control structure operably coupled to the at least one fabricated retort vessel at least partially disposed within the bore.

95. (canceled)

96. A system for extracting hydrocarbons from organic material, the system comprising:
a substantially vertical shaft defining a subterranean bore;
a process isolation barrier comprising a liner surrounding the shaft and in contact with a face of at least one earth formation and a top cap extending over an upper end of the shaft and comprising support structure suspending the top cap over the bore, a periphery of the top cap in substantially sealed engagement with the liner;
apparatus for introducing organic material into the upper end of the bore and configured for preventing substantial escape of vapor from the bore;
at least one fabricated preheat vessel supported within the bore and adapted to receive the organic material at least one fabricated retort vessel supported within the bore and adapted to receive the organic material from the at least one preheat vessel;
at least one fabricated cooling chamber supported within the bore adapted to receive the organic material from the at least one retort vessel;
at least one quenching chamber disposed within the bore and adapted to receive the organic material from the at least one fabricated cooling chamber;
apparatus for collecting hydrocarbons extracted from the organic material; and
control structure operably coupled to the at least one fabricated retort vessel at least partially disposed within the bore.
